(19) United States
(12) Patent Application Publication
(10) Pub. No.: US 2016/0228037 A1
(43) Pub. Date: Aug. 11, 2016

(54) HOMECARE ASTHMA MANAGEMENT

(71) Applicant: ORIDION MEDICAL 1987 LTD., Jerusalem (IL)

(72) Inventors: Yacov Bubis, Jerusalem (IL); David P. Besko, Thornton, CO (US); Paul S. Addison, Edinburgh (GB); Michal Ronen, Givat-Brenner (IL)

(21) Appl. No.: 14/617,966

(22) Filed: Feb. 10, 2015

(51) Int. Cl. A61B 5/083; A61B 5/00; A61B 5/1455; A61B 5/024; A61B 5/08; A61B 5/091

(52) U.S. Cl. CPC A61B 5/0836; A61B 5/0816; A61B 5/091; A61B 5/14551; A61B 5/02416; A61B 5/742; A61B 2503/08; A61B 2505/07

(57) ABSTRACT

Device and method for assessing an asthma status of a subject including monitoring a breath related parameter of a subject suffering from asthma, comparing the breath related parameter to a baseline parameter of the subject; determining a deviation of the breath related parameter from the baseline parameter; obtaining an input parameter; and assessing the asthma status of the subject, based on an integrated analysis of the input parameter and of the deviation of the breath related parameter from the baseline parameter.

### TECHNICAL FIELD

The present disclosure generally relates to the field of breath monitoring and asthma.

### BACKGROUND

Asthma is a common chronic inflammatory disease of the airways characterized by variable and recurring symptoms, reversible airflow obstruction and bronchospasm. Common symptoms include wheezing, coughing, chest tightness, and shortness of breath.

Asthma is thought to be caused by a combination of genetic and environmental factors. Its diagnosis is usually based on the pattern of symptoms, response to therapy over time and spirometry. It is clinically classified according to the frequency of symptoms, forced expiratory volume in one second (FEV1), and peak expiratory flow rate.

### SUMMARY

Aspects of the disclosure, in some embodiments thereof, relate to devices and methods for assessing the asthma status of a patient.

Monitoring asthma on a daily basis is recommended in subjects with moderate or severe persistent asthma and/or subjects with recurrent severe exacerbations. Studies have shown that monitoring asthma regularly enables a more controlled use of medications, decreased asthma exacerbations and decreased emergency room visits. Typically daily monitoring of asthma is performed by evaluating forced vital capacity (FVC) and forced expiratory volume in one second (FEV1) using spirometry and the ability of the lungs to push out air using peak flow measurements. However, using spirometry and peak flow measurements, mainly in children, is often complicated and the reliability of the measurements is consequently reduced. Similarly, performing spirometry and peak flow measurements under severe asthma conditions such as during an exacerbation is also a complex task.

Patients with asthma have higher exhaled nitric oxide (eNO) levels than other people. Monitoring eNO has therefore been suggested as an alternative to spirometry and peak flow measurements. However, to date, the results in both adults and children have been modest and this technique is currently not universally recommended, since other factors also influence eNO levels.

Advantageously, the device and method, disclosed herein, are configured to accurately assess the asthma status of a patient, based on at least one breath related parameter monitored using a capnograph and/or a pulse oximeter. Furthermore, the devise and method, disclosed herein may enable the identification and/or the prediction of an upcoming exacerbation, thereby allowing preemptive measures to be taken and avoiding deterioration. Furthermore, the method may enable monitoring follow-up responses to a prescribed medication and thereby facilitate identification of a preferred and/or a personalized asthma therapy.

As a further advantage, the method and device disclosed herein may be configured to incorporate input parameters relevant to the interpretation of the monitored breath related parameters. This enables a context sensitive evaluation of the monitored breath related parameters. For example, environmental conditions such as air quality may be provided as an input parameter enabling interpretation of the monitored breath related parameters in view of, for example, the degree of air pollution.

In addition, the device and method, disclosed herein, advantageously enable the formation of a personalized library of monitored breath related parameters. This again allows the determination of personalized baselines and/or threshold settings to which subsequently monitored breath related parameters may be compared, thereby facilitating determining subtle changes in the patient's asthma status.

The method and device disclosed herein are particularly suitable for home care asthma management.

According to some embodiments, there is provided a method for assessing an asthma status of a subject. According to some embodiments, the method includes monitoring at least one breath related parameter of a subject suffering from asthma for a predetermined period of time, using at least one sensing device; comparing the at least one breath related parameter to a baseline parameter of the subject; determining a deviation of the breath related parameter from the baseline parameter; obtaining at least one input parameter; and assessing the asthma status of the subject, based on an integrated analysis of the deviation of the at least one breath related parameter from the baseline parameter and of the at least one input parameter.

According to some embodiments, the input parameter may include: time of monitoring, type of medication, time of medication, dose of medication, air quality during monitoring or any combination thereof. According to some embodiments, the input parameter may be air quality during monitoring.

According to some embodiments, the baseline parameter may be an exacerbation threshold parameter. According to some embodiments, the baseline parameter may be determined based on a library of pre-stored parameters of the subject obtained during at least one previous monitoring session.

According to some embodiments, the method may further include predicting and/or identifying an exacerbation event in the subject based on the assessed asthma status.

According to some embodiments, the sensing device may be a capnograph. According to some embodiments, the at least one breath related parameter may include: a waveform, a representative waveform, a respiration rate, an inhalation to exhalation ratio (I:E ratio), end-tidal carbon dioxide (EtCO2) of a waveform and/or a representative waveform, upward slope of a waveform and/or a representative waveform, alpha angle of a waveform and/or a representative waveform, beta angle of a waveform and/or a representative waveform, or any combination thereof.

According to some embodiments, the sensing device may be a pulse oximeter. According to some embodiments, the at least one breath related parameter may include: saturation of peripheral oxygen (SpO2), pulse rate, pleth wave, respiratory effort, or any combination thereof.

According to some embodiments, the monitoring may be performed daily. According to some embodiments, the predetermined time of monitoring may be in the range of 2-10 minutes.

According to some embodiments, the method may further include adding the at least one breath related parameter to the library of pre-stored parameters; thereby generating an updated library.

According to some embodiments, the method may further include computing a trend in the at least one breath related parameter based on the library of pre-stored parameters.

According to some embodiments, the method may further include monitoring a fraction of exhaled nitric oxide (FeNO) in the subject's breath.

According to some embodiments, the assessment of the asthma status of the subject may further be based on the fraction of exhaled nitric oxide (FeNO) in the subject's breath.

According to some embodiments, the method may further include displaying the at least one breath related parameter, parameters deviating from baseline, a computed trend in the at least one breath related parameter and/or the assessed asthma status of the subject on a display.

According to some embodiments, the method may further include communicating the at least one breath related parameter, parameters deviating from baseline, a computed trend in the at least one breath related parameter and/or the assessed asthma status of the subject to a remote computer and/or a caregiver.

According to some embodiments, the subject is a child.

According to some embodiments, there is provided a computing device including a processor, the processor configured to receive at least one breath related parameter of an asthma subject; compare the at least one breath related parameter to a baseline parameter of the subject; determine a deviation of the breath related parameter from the baseline parameter; obtain at least one input parameter; and assessing the asthma status of the subject, based on an integrated analysis of the deviation of the at least one breath related parameter from the baseline parameter and of the at least one input parameter.

According to some embodiments, the input parameter may include: time of monitoring, type of medication, time of medication, dose of medication, air quality during monitoring or any combination thereof.

According to some embodiments, the processor may further be configured to predict and/or identify an exacerbation event in the asthma subject.

According to some embodiments, the computing device may further include a display configured to display the at least one breath related parameter, parameters deviating from baseline, a computed trend in the at least one breath related parameter and/or the assessed asthma status of the subject.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

### BRIEF DESCRIPTION OF THE DRAWINGS

Some embodiments of the disclosure are described herein with reference to the accompanying figures. The description, together with the figures, makes apparent to a person having ordinary skill in the art how some embodiments of the disclosure may be practiced. The figures are for the purpose of illustrative discussion and no attempt is made to show structural details of an embodiment in more detail than is necessary for a fundamental understanding of the teachings of the disclosure. For the sake of clarity, some objects depicted in the figures are not to scale.

FIG. 1 schematically shows a normal CO₂ waveform according to some embodiments;

FIG. 2 schematically shows waveforms obtained in asthma patients as compared to a normal CO₂ waveform, according to some embodiments;

FIG. 3 is an illustrative flowchart depicting the method for assessing a subject's asthma, according to some embodiments;

FIG. 4 is an illustrative flowchart depicting the method for assessing a subject's asthma, according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

There is provided, according to some embodiments, a method for assessing an asthma status of a patient. According to some embodiments, the method may include monitoring at least one breath related parameter of an asthma patient for a predetermined period of time, using at least one sensing device. The method further includes comparing the at least one monitored breath related parameter to a baseline parameter of the patient and determining a deviation of the monitored breath related parameter from the baseline parameter. The asthma status of the patient may then be assessed based on an integrated analysis of the deviation (and/or degree of deviation) of the at least one monitored breath related parameter from the baseline parameter and of at least one additional input parameter.

As referred to herein, the terms “patient” and “subject” may interchangeably be used and may relate to a subject suffering from asthma. According to some embodiments, the subject may be an infant, a child, an adolescence, an adult or an elderly. Each possibility is a separate embodiment. According to some embodiments, the subject may be cognitively disabled. According to some embodiments, the subject may be unable to follow written and/or vocal instructions.

According to some embodiment, the assessment of the subject's asthma status may be based on discontinues monitoring sessions. According to some embodiments, the subject may undergo weekly, daily and/or hourly monitoring sessions to assess his or hers asthma status and/or to identify deteriorations/improvements in the subjects conditions. Each possibility is a separate embodiment. According to some embodiments, the method may be configured for use in home-care asthma management.

According to some embodiments, each monitoring session may have a duration of 1-30 minutes, 1-10 minutes, 1-5 minutes, 2-5 minutes or any other suitable time duration within the range of 1-30 minutes. Each possibility is a separate embodiment.

According to some embodiments, a monitoring session may include 1-100, 1-50, 1-25, 2-20, 2-10 breaths or any other suitable number of breaths within the range of 1-100 breaths. Each possibility is a separate embodiment. According to some embodiments, the breaths may be deep breaths. According to some embodiments, the breaths may be regular breaths.

According to some embodiments, the sensing device may be a capnograph and/or a pulse oximeter.

According to some embodiments, the at least one breath related parameter may include a parameter obtained and/or derived from a capnograph, such as but not limited to a waveform, a representative waveform, a respiration rate, an inhalation to exhalation ratio (I:E ratio), end-tidal carbon dioxide (EtCO₂) of a waveform and/or a representative waveform, upward slope of a waveform and/or a representative waveform, alpha angle of a waveform and/or a representative waveform, beta angle of a waveform and/or a representative waveform, or any combination thereof. Each possibility is a separate embodiment.

According to some embodiments, the at least one breath related parameter may include a PPG signal, such as but not limited to saturation of peripheral oxygen (SpO2), pulse rate, pleth wave, respiratory effort, or any combination thereof. Each possibility is a separate embodiment. As used herein the term “PPG signal” may refer to the signal obtained and/or derived from a oximeter such as for example a pulse oximeter configured to determine the oxygen saturation of the blood.

As used herein the terms “effort”, “breathing effort” and “respiratory effort” interchangeably refer to physical effort or work of a process, such as for example effort of breathing. The respiratory effort may in turn affect respiratory signals, such as, but not limited to, a PPG signal. Respiratory effort may increase, for example, if a patient's respiratory pathway becomes restricted or blocked. Conversely, respiratory effort may decrease as a patient's respiratory pathway becomes unrestricted or unblocked. According to some embodiments, the respiratory effort may be derived from a PPG signal.

According to some embodiments, the at least one breath related parameter may include an algorithmically-derived index of multiple parameters. According to some embodiments, the multiple parameters may at least be obtained from a capnograph and a pulse oximeter. According to some embodiments, the multiple parameters may further be obtained from a spirometer, a peak flow measurement device and/or eNO measurement device. Each possibility is a separate embodiment. According to some embodiments, each of the multiple parameters may be obtained during a same or a different monitoring session. According to some embodiments, the algorithmically-derived index of multiple parameters may be computed by:

(a) characterizing a first measured medical parameter based on a comparison of the first measured medical parameter against a first reference value;

(b) characterizing a second measured medical parameter based on a comparison of the second measured medical parameter against a second reference value; and

(c) computing the index value based on values associated with each of the characterized first and second measured medical parameters.

As used herein, the term “at least one” when referring to monitored breath related parameters may include 1, 2, 3, 4, 5, 10 or more parameters. Each possibility is a separate embodiment. According to some embodiments, the breath related parameters may be obtained from a same or a different sensing device.

As used herein the term “baseline parameter” may refer to a reference value to which the monitored breath related parameter is compared. According to some embodiments, the baseline parameter may be a textbook parameter indicative of a normal condition. According to some embodiments, the baseline parameter may be a textbook parameter indicative of an asthma exacerbation. According to some embodiments, the baseline parameter may be a reference value obtained from the (same) patient when being devoid of asthmatic symptoms. According to some embodiments, the baseline parameter may be a reference value obtained from the (same) patient during an asthma exacerbation. According to some embodiments, the baseline parameter may be a reference value calculated from a plurality of monitoring sessions of the patient when being devoid of asthmatic symptoms and/or during an asthma exacerbation. According to some embodiments, the baseline parameter may be updated after each monitoring session based on the newly monitored parameters.

According to some embodiments, the integrated analysis of the deviation of the at least one monitored breath related parameter from the baseline parameter and of the at least one input parameter, may include weighting the determined deviation according to the received input parameter. For example, an abnormal CO₂ waveform obtained when air pollution is high may be indicative of a coming deterioration in the patient's asthma status. Accordingly, deviations obtained during high air pollution may receive a higher weight than a similar abnormal parameters obtained when air pollution is low. Similarly, deviations obtained following medication may receive a higher weight than a similar abnormal parameters obtained without medication.

As used herein, the term “at least one” may refer to 1, 2, 3, 4, 5, 10 or more. Each possibility is a separate embodiment. As used herein, the term “at least two” may refer to 2, 3, 4, 5, 10 or more. Each possibility is a separate embodiment.

As used herein, the term “plurality” when referring to monitoring sessions may include 2, 3, 4, 5, 10, 20, 50 or more monitoring sessions. Each possibility is a separate embodiment.

According to some embodiments, the method may further enable predicting and/or identifying an exacerbation event in the subject based on the assessed asthma status. As used herein the term “asthma exacerbation” may refer to an asthma attack during which the airways become swollen and inflamed and the muscles around the airways contract, causing breathing (bronchial) tubes to narrow. It is thus understood that by predicting/anticipating the exacerbation and/or identifying the exacerbation at an early step thereof may enable preemptive treatments which may avert further deterioration. Additionally or alternatively, if a severe asthma attack is identified, the method may provide an indication that medical attention is required.

According to some embodiments, the method may enable the formation of a personalized library of monitored breath related parameters. This again may allow the determination of personalized baselines and/or threshold settings to which subsequently monitored parameters may be compared. The personalized baseline and/or threshold settings may facilitate determining even subtle changes in the patient's asthma status. Moreover, the personalized baseline and/or threshold settings may enable to determine progression, deterioration or improvement of the asthmatic condition. According to some embodiments, the method may include computing a trend in the at least one monitored breath related parameter based on the library of pre-stored parameters.

According to some embodiments, the time period between subsequent monitoring sessions may be constant, for example, once every day. According to some embodiments, the time period between subsequent monitoring sessions may be variable. According to some embodiments, the method may provide an indication of a desired time for a subsequent monitoring session, based on the assessed asthma status. As a non-limiting example, if the at least one monitored breath related parameter, the trend therein crosses a pre-determined threshold value and/or is indicative of deterioration in the patient's asthma status, the method may provide an indication that a subsequent monitoring session is desired within a time frame shorter than if normal values are obtained, for example within a few hours. As another non-limiting example, if the assessed asthma status is indicative of a normal breath status, the subsequent monitoring session may be postponed to the next day.

According to some embodiments, the method may include displaying the at least one monitored breath related parameter, parameters deviating from baseline, a computed trend in the at least one monitored parameter and/or the assessed asthma status of the subject on a display. Each possibility is a separate embodiment.

According to some embodiments, the method may include saving the at least one monitored breath related parameter, parameters deviating from baseline, a computed trend in the at least one monitored parameter and/or the assessed asthma status of the subject with an indication of the time and/or date of the monitoring session. Each possibility is a separate embodiment. This may enable off-line correlation of the monitored parameter and/or the library of monitored parameters to additional input parameters, such as time of day, weather, air quality, season and the like. Each possibility is a separate embodiment. According to some embodiments, the method may include updating the at least one input parameter (upon which the assessment of the subject's asthma status is relied) based on a detected/identified correlation. According to some embodiments, the method may include adding at least one additional input parameter (upon which the assessment of the subject's asthma status is relied) based on a detected/identified correlation.

According to some embodiments, the method may include communicating the at least one monitored breath related parameter, parameters deviating from baseline, a computed trend in the at least one monitored parameter and/or the assessed asthma status of the subject to a remote computer and/or a caregiver. Each possibility is a separate embodiment.

According to some embodiments, the method may further include monitoring a fraction of exhaled nitric oxide (FeNO) in the subject's breath. According to some embodiments, the assessment of the subject's asthma status may further be based on the fraction of exhaled nitric oxide (FeNO) in the subject's breath. According to some embodiments, incorporating FeNO readings into the assessment of the subject's asthma status may enable reducing the number of required monitoring sessions. According to some embodiments, monitoring FeNO in the subject's breath may be performed when the assessed asthma status and/or the trend therein (determined according to the method disclosed herein) is indicative of a deterioration in the patient's status. Alternatively, the method disclosed herein may be supplemental to asthma monitoring based on FeNO readings. For example, if FeNO readings are indicative of deterioration in the patient's asthma status, the method disclosed herein may provide a further indication reaffirming or refuting the FeNO readings, thereby providing a more reliable assessment of the patient's asthma status.

According to some embodiments, there is provided a method including monitoring FeNO in a breath of a subject suffering from asthma, comparing the monitored FeNO to a predetermined baseline value, and monitoring at least one CO₂ parameter of the subject when a deviation in the monitored FeNO, from the predetermined baseline, crosses a threshold value.

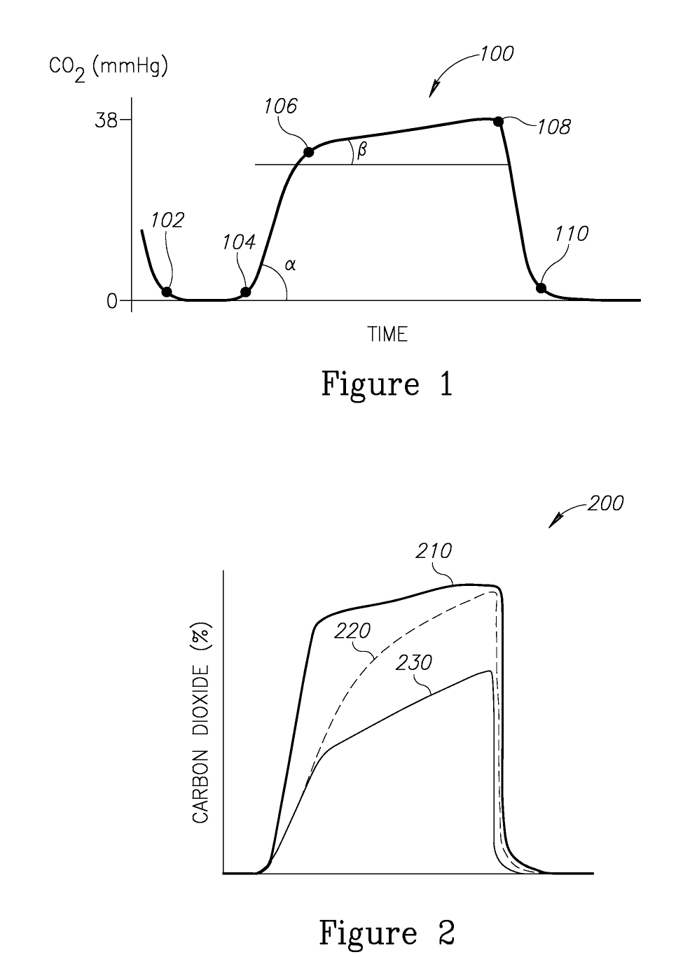

Reference is now made to FIG. 1, which shows an adult normal capnogram 100 as known in the art. Adult normal capnogram 100 in spontaneously breathing subjects may be characterized by four distinct phases:
1. Dead space ventilation: Shown between points 102 and 104 in the figure, this is the earliest phase of exhalation. Physiologally, this phase corresponds to initial exhalation from upper airway (mainstem bronchi, trachea, posterior pharynx, mouth and nose).
2. Ascending phase: Shown between points 104 and 106 is a rapid rise in CO₂ concentration, which physiologically corresponds to alveolar gas reaching the upper airways.
3. Alveolar plateau: Shown between points 106 and 108, this is the stage where CO₂ reaches a generally steady state, sometimes having a mild ascending slope. Physiologally, this phase corresponds to a uniform CO₂ level attained in the entire breath stream.
4. Inspiratory limb: Shown between points 108 and 110 is a rapid decrease in CO2 concentration back to zero, marking the beginning of an inhalation.

Point 108, which is the intersection of the alveolar plateau and the inspiratory limb, is often referred to as the End-Tidal CO₂ (EtCO₂).

An angle α (alpha), which designates the angle between the ascending phase curve and the X axis, is referred to as a “takeoff angle”. An angle β (beta), which designates the angle between the alveolar plateau and the X axis, is referred to as an “elevation angle”.

An amplitude of capnogram 100 is dependent on EtCO₂ concentration. A width of capnogram 100 is dependent on expiratory time. The shape of capnogram 100 is generally rectangular, formed by almost perpendicular ascending phase (indicating absence of lower airway obstruction) and inspiratory limb (no upper airway obstruction).

Reference is now made to FIG. 2, which shows exemplary waveforms 200 which may be obtained from a subject depending on his/hers asthma status. Waveform 210 represents a normal capnogram. Waveform 220 represent a capnogram obtained from a subject having an obstructed upper airway, such as during an asthma attack. The asthma attack may be relatively mild as in waveform 220 or be indicative of severe airway obstruction, as in waveform 230.

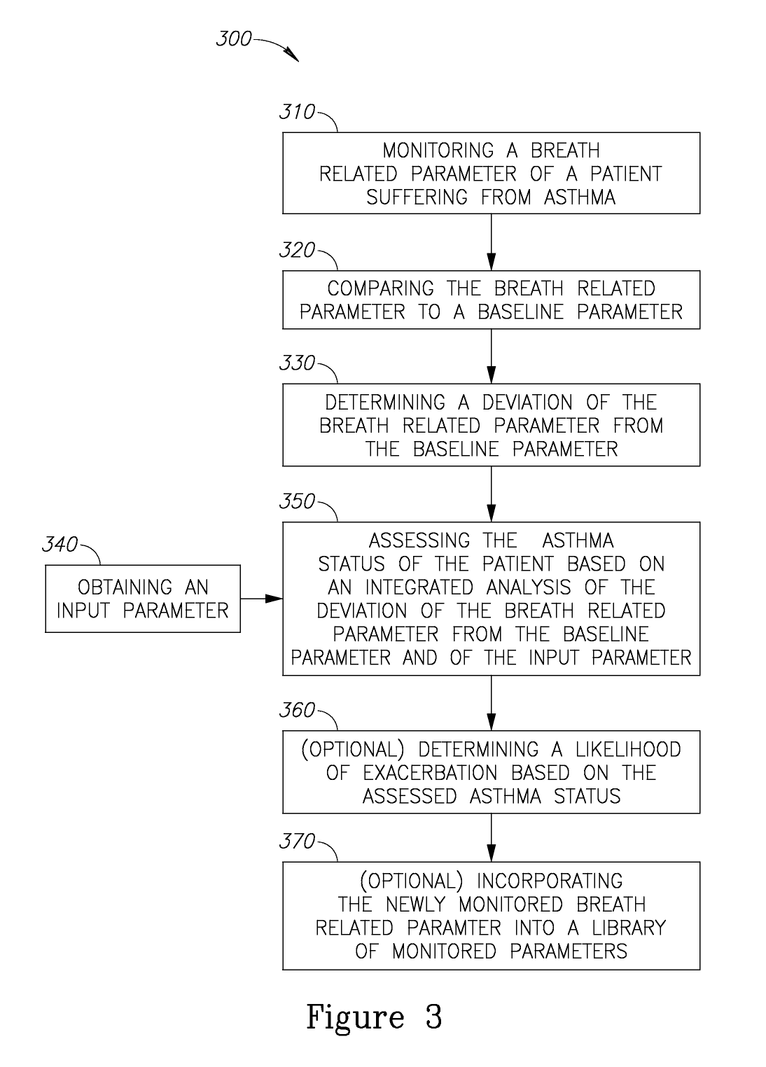

Reference is now made to FIG. 3, which is an illustrative flowchart 300 depicting the method for assessing a subject's asthma, according to some embodiments. It is understood by one of ordinary skill of the art that the order of the methods as described should not be construed as sequential steps, and a different sequence of events may be envisaged.

In step 310, a breath related parameter of an asthma patient is monitored for a predetermined period of time, For example, the CO₂ level of the patient may be monitored using a capnograph for approximately 5 minutes during a first monitoring session. At step 320, the monitored breath related parameter is compared to a baseline parameter of the patient. For example, the monitored CO₂ waveform may be compared to a normal “textbook” waveform. Additionally or alternatively, the monitored waveform (or other parameter) may be compared to a subject specific reference waveform. The subject specific reference waveform may be representative of the subject's normal waveform or of a waveform obtained during an asthma exacerbation. Additionally or alternatively, the baseline waveform may be a waveform computed from a plurality of monitoring sessions of the subject. In step 330, a deviation of the monitored parameter from the baseline parameter is determined. In step 340, an input parameter, such as, but not limited to, a value indicative of the degree of air pollution is obtained. It is understood, that the input parameter may be directly monitored/determined and or retrieved from websites, mobile applications or any other suitable information source. It is further understood, that the input parameter may be obtained, prior to, simultaneous with or subsequently to the monitoring of the breath related parameter. Each possibility is a separate embodiment. In step 350, the asthma status of the patient is assessed, based on an integrated analysis of the deviation of the breath related parameter from the baseline parameter and of the input parameter. Optionally, in step 360 the likelihood of a forthcoming exacerbation may be determined based on the assessed asthma status. It is understood that following steps 350 or 360, the method may be repeated for a second monitoring session. In addition, in an optional step 370, the newly monitored breath related parameters may be incorporated into a library of monitored parameters, as essentially described herein.

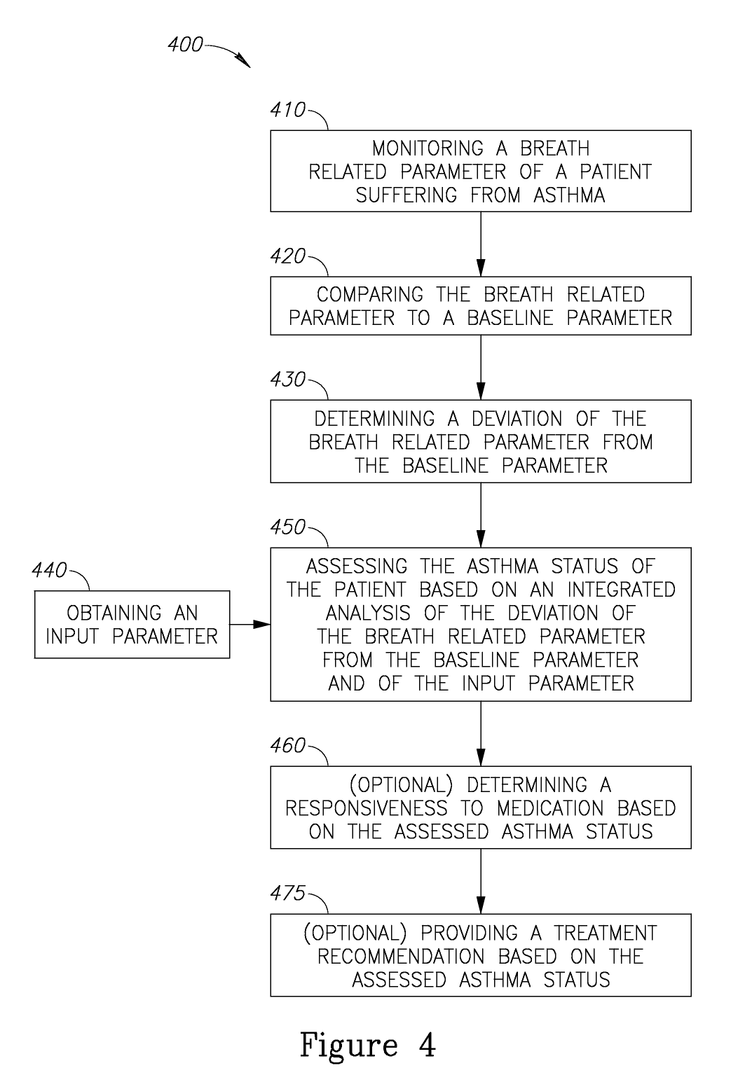

Reference is now made to FIG. 4, which is an illustrative flowchart 400 depicting the method for assessing a subject's asthma, according to some embodiments. It is understood by one of ordinary skill of the art that the order of the methods as described should not be construed as sequential steps, and a different sequence of events may be envisaged.

In step 410, a breath related parameter of an asthma patient is monitored for a predetermined period of time, For example, the respiratory effort of the patient may be monitored using a pulse oximeter for approximately 5 minutes during a first monitoring session. At step 420, the breath related parameter is compared to a baseline parameter of the patient. As a non-limiting example, the monitored respiratory effort may be compared to a normal respiratory effort value. Additionally or alternatively, the monitored respiratory effort may be compared to a subject specific reference respiratory effort. The subject specific reference waveform may be representative of the subject's normal respiratory effort or of a respiratory effort obtained during an asthma exacerbation. Additionally or alternatively, the baseline respiratory effort may be a respiratory effort computed from a plurality of monitoring sessions of the subject. In step 430, a deviation of the monitored breath related parameter from the baseline parameter is determined. In step 440, an input parameter, such as, but not limited to, time and/or type of medication is obtained. It is understood, that the input parameter may be obtained, prior to, simultaneous with or subsequently to the monitoring of the breath related parameter. Each possibility is a separate embodiment. In step 450, the asthma status of the patient is assessed, based on an integrated analysis of the input parameter and of the deviation in the monitored breath related parameter from the baseline parameter. Optionally, in step 460 the responsiveness of the subject to the medication may be determined. For example, if an improvement in the monitored parameter is determined in response to the medication taken, improvement in the subject's asthma status may be determined and/or an exacerbation alert may be avoided. Alternatively, devoid a positive change in the subject's asthma status, despite medications taken, may serve as an indication/predication of an upcoming severe exacerbation. Based on the determined asthma status a recommendation may be provided in an additional optional step 475. Optional recommendations include increasing dosage of medication, changing type of medication, medical attention required or any other suitable recommendation or combination thereof. Each possibility is separate embodiment. In addition, the newly monitored breath related parameters may be incorporated into a library of monitored parameters, as essentially described herein.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising”, when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

1. A method for assessing an asthma status of a subject, the method comprising:
monitoring at least one breath related parameter of a subject suffering from asthma for a predetermined period of time, using at least one sensing device;
comparing the at least one breath related parameter to a baseline parameter of the subject;
determining a deviation of the breath related parameter from the baseline parameter;
obtaining at least one input parameter, the input parameter comprising: time of monitoring, type of medication, time of medication, dose of medication, air quality during monitoring or any combination thereof; and
assessing the asthma status of the subject, based on an integrated analysis of the deviation of the at least one breath related parameter from the baseline parameter and of the at least one input parameter.

2. The method of claim 1, further comprising predicting and/or identifying an exacerbation event in the subject based on the assessed asthma status.

3. The method of claim 2, wherein the baseline parameter is an exacerbation threshold parameter.

4. The method of claim 1, wherein the sensing device is a capnograph.

5. The method of claim 4, wherein the at least one breath related parameter comprises: a waveform, a representative waveform, a respiration rate, an inhalation to exhalation ratio (I:E ratio), end-tidal carbon dioxide (EtCO₂) of a waveform and/or a representative waveform, upward slope of a waveform and/or a representative waveform, alpha angle of a waveform and/or a representative waveform, beta angle of a waveform and/or a representative waveform, or any combination thereof.

6. The method of claim 1, wherein the sensing device is a pulse oximeter.

7. The method of claim 6, wherein the at least one breath related parameter comprises: saturation of peripheral oxygen (SpO₂), pulse rate, pleth wave, respiratory effort, or any combination thereof.

8. The method of claim 1, wherein the at least one input parameter comprises air quality during monitoring.

9. The method of claim 1, wherein the monitoring is performed daily and wherein the predetermined time of monitoring is in the range of 2-10 minutes.

10. The method of claim 1, wherein the baseline parameter is determined based on a library of pre-stored parameters of the subject obtained during at least one previous monitoring session.

11. The method of claim 10, further comprising adding the at least one breath related parameter to the library of pre-stored parameters; thereby generating an updated library.

12. The method of claim 10, further comprising computing a trend in the at least one breath related parameter based on the library of pre-stored parameters.

13. The method of claim 1, further comprising monitoring a fraction of exhaled nitric oxide (FeNO) in the subject's breath.

14. The method of claim 13, wherein the assessment of the asthma status of the subject is further based on the fraction of exhaled nitric oxide (FeNO) in the subject's breath.

15. The method of claim 1, further comprising displaying the at least one breath related parameter, parameters deviating from baseline, a computed trend in the at least one breath related parameter and/or the assessed asthma status of the subject on a display.

16. The method of claim 1, further comprising communicating the at least one breath related parameter, parameters deviating from baseline, a computed trend in the at least one breath related parameter and/or the assessed asthma status of the subject to a remote computer and/or a caregiver.

17. The method of claim 1, wherein the subject is a child.

18. A computing device comprising a processor configured to:
receive at least one breath related parameter of an asthma subject;
compare the at least one breath related parameter to a baseline parameter of the subject;
determine a deviation of the breath related parameter from the baseline parameter;
obtain at least one input parameter, the input parameter comprising: time of monitoring, type of medication, time of medication, dose of medication, air quality during monitoring or any combination thereof; and
assessing the asthma status of the subject, based on an integrated analysis of the deviation of the at least one breath related parameter from the baseline parameter and of the at least one input parameter.

19. The computing device of claim 18, wherein said processor is further configured to predict and/or identify an exacerbation event in said asthma subject.

20. The computing device of claim 18, further comprising a display configured to display the at least one breath related parameter, parameters deviating from baseline, a computed trend in the at least one breath related parameter and/or the assessed asthma status of the subject.
